(19) United States
(12) Patent Application Publication
(10) Pub. No.: US 2010/0063448 A1
(43) Pub. Date: Mar. 11, 2010

(54) PUMP ASSEMBLY COMPRISING ACTUATOR SYSTEM

(75) Inventors: Karsten Randrup Kragh, Copenhagen (DK); Finn Jensen, Hinnerup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/529,882

(22) PCT Filed: Mar. 6, 2008
(86) PCT No.: PCT/EP2008/052707

§ 371 Date: Nov. 12, 2009

(30) Foreign Application Priority Data
Mar. 6, 2007 (EP) 07103590.1

(51) Int. Cl. A61M 5/142; F04B 43/00; F04B 43/04

(52) U.S. Cl. 604/153; 417/437; 417/412

(57) ABSTRACT

The invention provides a pump assembly comprising an actuator lever, an actuator for moving the actuator lever, a supporting structure, a pump comprising a pump member moveable by actuation of the actuator lever, a first pivoting joint formed between the actuator lever and the supporting structure, and a second pivoting joint formed between the actuator and the supporting structure. The actuator lever and the actuator are coupled to each other by a coupling joint arranged between the first and the second pivoting joint in such a way that rotation of the actuator in a first direction causes the actuator lever to rotate in an opposite second direction. By providing an actuator system comprising two actuator elements linked to each other by a coupling joint ensuring counter rotation of the two members, a system is provided which can be made less susceptible to the influence of acceleration.

The present invention relates to an actuator system suitable for actuation of pumps for the delivery of fluids. In a specific aspect, the invention relates to an actuator system suitable for actuating a membrane pump arranged in a drug delivery device adapted to be carried by a person. However, the present invention may find broad application in any field in which a given member, component or structure is to be moved in a controlled manner.

### BACKGROUND OF THE INVENTION

In the disclosure of the present invention reference is mostly made to the treatment of diabetes by injection or infusion of insulin, however, this is only an exemplary use of the present invention.

Portable drug delivery devices for delivering a drug to a patient are well known and generally comprise a reservoir adapted to contain a liquid drug and having an outlet in fluid communication with a transcutaneous access device such as a hollow infusion needle or a cannula, as well as expelling means for expelling a drug out of the reservoir and through the skin of the subject via the access device. Such drug delivery devices are often termed infusion pumps.

Basically, infusion pumps can be divided into two classes. The first class comprises infusion pumps which are relatively expensive pumps intended for 3-4 years use, for which reason the initial cost for such a pump often is a barrier to this type of therapy. Although more complex than traditional syringes and pens, the pump offer the advantages of continuous infusion of insulin, precision in dosing and optionally programmable delivery profiles and user actuated bolus infusions in connections with meals.

Addressing the above problem, several attempts have been made to provide a second class of drug infusion devices that are low in cost and convenient to use. Some of these devices are intended to be partially or entirely disposable and may provide many of the advantages associated with an infusion pump without the attendant cost and inconveniences, e.g. the pump may be prefilled thus avoiding the need for filling or refilling a drug reservoir. Examples of this type of infusion devices are known from U.S. Pat. Nos. 4,340,048 and 4,552,561 (based on osmotic pumps), U.S. Pat. No. 5,858,001 (based on a piston pump), U.S. Pat. No. 6,280,148 (based on a membrane pump), U.S. Pat. No. 5,957,895 (based on a flow restrictor pump, also known as a bleeding hole pump), U.S. Pat. No. 5,527,288 (based on a gas generating pump), or U.S. Pat. No. 5,814,020 (based on a swellable gel) which all in the last decades have been proposed for use in inexpensive, primarily disposable drug infusion devices, the cited documents being incorporated by reference.

As the membrane pump can be used as a metering pump (i.e. each actuation (or stroke) of the pump results in movement of a specific amount of fluid being pumped from the pump inlet to the pump outlet side) a small membrane pump would be suitable for providing both a basal drug flow rate (i.e. providing a stroke at predetermined intervals) as well as a drug bolus infusion (i.e. a given number of strokes) in a drug delivery device of the above-described type.

More specifically, a metering membrane pump may function as follows. In an initial condition the pump membrane is located at an initial predefined position and the inlet and outlet valves are in their closed position. When the means for moving the membrane (i.e. the membrane actuator) is energized an increase of the pressure inside the pumping chamber occurs, which causes opening of the outlet valve. The fluid contained in the pumping chamber is then expelled through the outflow channel by the displacement of the pump membrane from its initial position towards a fully actuated position corresponding to the end position for the “out-stroke” or “expelling-stroke”. During this phase, the inlet valve is maintained closed by the pressure prevailing in the pumping chamber. When the pump membrane is returned to its initial position (either due to its elastic properties or by means of the membrane actuator) the pressure in the pumping chamber decreases. This causes closing of the outlet valve and opening of the inlet valve. The fluid is then sucked into the pumping chamber through the in-flow channel, owing to the displacement of the pump membrane from the actuated position to the initial position corresponding to the end position for the “in-stroke” or “suction-stroke”. As normally passive valves are used, the actual design of the valve will determine the sensitivity to external conditions (e.g. back pressure) as well as the opening and closing characteristics thereof, typically resulting in a compromise between the desire to have a low opening pressure and a minimum of backflow. As also appears, a metering membrane functions as any conventional type of membrane pump, for example described for use as a fuel pump in U.S. Pat. No. 2,980,032.

As follows from the above, the precision of a metering pump is to a large degree determined by the pump membranes movement between its initial and actuated positions in a controlled manner. For example, movement may be determined by a membrane actuator member being moved between predefined positions as disclosed in WO 2005/094919. More specifically, in such a prior art pump assembly a pump actuator is provided in the form of a pivoting actuator lever acting on a pump piston, the actuator lever providing a coil-magnet actuator with the coil being arranged on the actuator lever and the magnets being arranged on a supporting structure. As the actuator lever has a pivoting point at one end of the lever and the relatively heavy coil is arranged at the other end of the lever, the lever is not balanced in respect of influences from the outside, i.e. if the pump and its supporting structure is moved by external forces the lever will tend not to move with the pump but relative to the pump and thereby potentially actuate the pump, this due to the momentum of inertia of the lever.

Having regard to the above-identified problems, it is an object of the present invention to provide an actuator system, or component thereof, suitable for driving an actuatable structure or component in a controlled manner and being adapted to withstand external influences to a higher degree than known systems.

It is a further object to provide an actuator system which can be used in combination with a pump assembly arranged in a portable drug delivery device, system or a component therefore, thereby providing controlled infusion of a drug to a subject. It is a further object to provide an actuator system which can be used in combination with a pump such as a membrane pump. It is a further object of the invention to provide an actuator, or component thereof, which can be provided and applied in a cost-effective manner.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, the present invention provides a pump assembly comprising an actuator lever, an actuator comprising an actuator member for moving the actuator lever, a supporting structure, a pump comprising a pump member moveable by actuation of the actuator lever, a first pivoting joint formed between the actuator lever and the supporting structure, and a second pivoting joint formed between the actuator member and the supporting structure. The actuator lever and the actuator member are coupled to each other by a coupling joint arranged between the first and the second pivoting joint in such a way that rotation of the actuator member in a first direction causes the actuator lever to rotate in an opposite second direction.

By providing an actuator system comprising two actuator elements linked to each other by a coupling joint ensuring counter rotation of the two elements, a system is provided which can be made less susceptible to the influence of acceleration.

In an exemplary embodiment the coupling joint provides a variable gear ratio for the translation of rotational movement from the actuator member to the actuator lever. The coupling joint may comprise a pin and a guide slot in which the pin is arranged to slide, wherein the position of the pin in the guide slot determines the actual gear ratio between the actuator lever and the actuator member. The actuator lever may be moved between a first position and a second position, the assembly comprising first and second stop means adapted to restrict movement of the actuator lever in the first respectively the second position. The stop means may be arranged on the supporting structure and may be adapted to engage the actuator lever in the first respectively the second position.

In an exemplary embodiment the pump member has a first position corresponding to the first position of the actuator lever and a first resting condition of the pump, and a second position corresponding to the second position of the actuator lever and a second actuated condition of the pump, wherein the pump member exerts a first force on the actuator lever in the first position and exerts a second higher force on the actuator lever in the second position. The pump may comprise a flexible member which is stretched by the pump member when the pump member is moved between its first and second positions, the pump member then exerting a larger force on the actuator lever when in the second position. To adjust to this situation the coupling joint may be designed to provide a first gear ratio when the actuator lever is in the first position and a second lower gear ratio when the actuator lever is in the second position. In the present context the term “gear ratio” is used to describe the actuator member's ability to transfer torque to the actuator lever, such that a low gear ratio means that the ability to transfer torque is high. In other words, in the initial position the actuator member has a lower ability to transfer torque.

The actuator may be of any suitable type, e.g. a coil-magnet actuator with the coil and magnet(s) being arranged on the actuator member respectively the supporting structure. As appears from the above, the term actuator is used to denote a system which only represents a part of a complete actuator. Indeed, a complete working actuator system would comprise additional components such as a controller and an energy source.

The pump may be adapted to pump a liquid between an inlet and an outlet, the pump member performing a pump stroke when actuated by the actuator lever. The pump may comprise inlet and outlet valves, e.g. membrane valves, associated with the pump inlet respectively the pump outlet, and a pump chamber actuated by the pump member to perform a pump stroke respectively a suction stroke. The assembly may further comprise a reservoir adapted to contain a fluid drug and comprising an outlet in fluid communication with or being adapted to be arranged in fluid communication with the pump inlet, and a transcutaneous access device comprising a distal end adapted to be inserted through the skin of a subject, the transcutaneous access device comprising an inlet in fluid communication with or being adapted to be arranged in fluid communication with the pump outlet. The pump assembly may be modified as desired, e.g. the pump may be programmable as well as wirelessly controlled, the reservoir may be prefilled with a drug and the transcutaneous access device may be actuatable from a retracted to an extended position. The balanced actuator system of the present invention may also be used in combinations with components other than a pump, e.g. an element to be moved may be arranged directly on the actuator lever.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals (including peptides, proteins, and hormones), biologically derived or active agents, hormonal and gene based agents, nutritional formulas and other substances in both solid (dispensed) and liquid form. In the description of the exemplary embodiments reference will be made to the use of insulin. Correspondingly, the term “subcutaneous” infusion is meant to encompass any method of parenteral delivery to a subject.

### BRIEF DESCRIPTION OF THE DRAWINGS

In the following the invention will be further described with references to the drawings, wherein

FIG. 1 shows an exploded view of an embodiment of a prior art actuator in combination with a pump,

FIG. 2 shows a schematic cross-sectional view through a pump and actuator assembly,

FIG. 3 shows a further prior art actuator,

FIG. 4 shows a cross-sectional view of the actuator of FIG. 3,

FIGS. 5 and 6 show an actuator system in a first respectively a second position,

FIG. 7 shows a pump unit with a pump assembly,

FIGS. 8 and 9 show a patch unit with a pump unit partly respectively fully attached, and

FIGS. 10A-10C show a lever and coil used for mathematical modelling.

In the figures like reference numerals are used to mainly denote like or similar structures.

### DESCRIPTION OF EXEMPLARY EMBODIMENTS

When in the following terms as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as their relative dimensions are intended to serve illustrative purposes only.

More specifically, a pump actuator 1 comprises an upper housing member 10 and a lower housing member 20, both comprising a distal main portion 11, 21 and a there from extending proximal arm portion 12, 22. On an upper surface of the lower main portion a pair of opposed walls 23, 24 are arranged and at the proximal end of the lower arm a post member 25 and a knife-edge member 26 are arranged perpendicularly to the general plane of the lower arm. In an assembled state the two main portions form a housing in which a pair of magnets 40, 41 is arranged on the opposed upper and lower inner surfaces of the main portions. The pump actuator further comprises a lever (or “arm”) 30 having a proximal end 31 comprising first and second longitudinally offset and opposed joint structures in the form of a groove 33 and a knife-edge 34 arranged perpendicular to a longitudinal axis of the lever, and a distal end 32 with a pair of gripping arms 35 for holding a coil member 36 wound from a conductor. A membrane pump is arranged in a pump housing 50 having a bore in which an actuation/piston rod 51 is arranged, the rod serving to actuate the pump membrane of the membrane pump (see below for a more detailed description of a membrane pump). The outer free end of the rod is configured as a substantially planar surface 52. In an assembled state the lever is arranged inside the housing with the coil positioned between the two magnets, and the housing is attached to the pump housing with the knife-edge of the knife-edge member 26 nested in the lever groove 33 and the knife-edge of the lever is positioned on the planar rod end surface, this arrangement providing first and second pivoting joints. As the actuating rod is biased outwardly by the elastic pump membrane the lever is held in place by the two joints and the housing in combination, the lever only being allowed to pivot relative to the first joint (see also below). Due to this arrangement a gearing of the force provided from the coil-magnet actuator to the actuation rod is realized, the gearing being determined by the disfence between the two pivoting joints (i.e. a first actuator arm) and the distance between the first/proximal pivoting joint and the “effective” position of the coil on the lever (i.e. a second actuator arm). By the term “effective”, the issue is addressed that the force generated by the coil actuator may vary as a function of the rotational position of the lever, this being due to the fact that the coil is moved between stationary magnets, which may result in a varying magnetic field for the coil as it is moved. The actuator further comprises a pair of contact members 28, 29 adapted to cooperate with a contact rod 37 mounted in the housing. In respect of the contact members and their use to monitor operation of a pump assembly reference is made to applicants co-pending application WO 2005/094919.

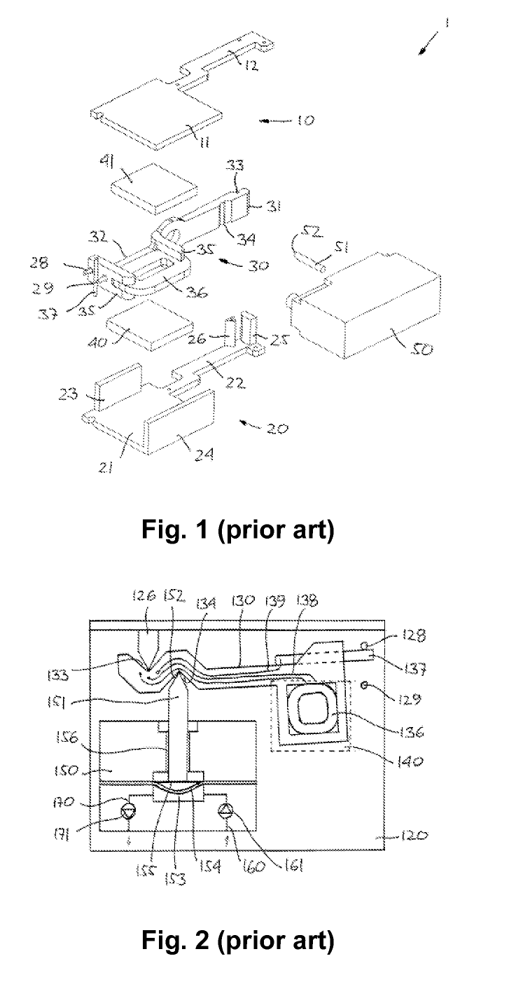

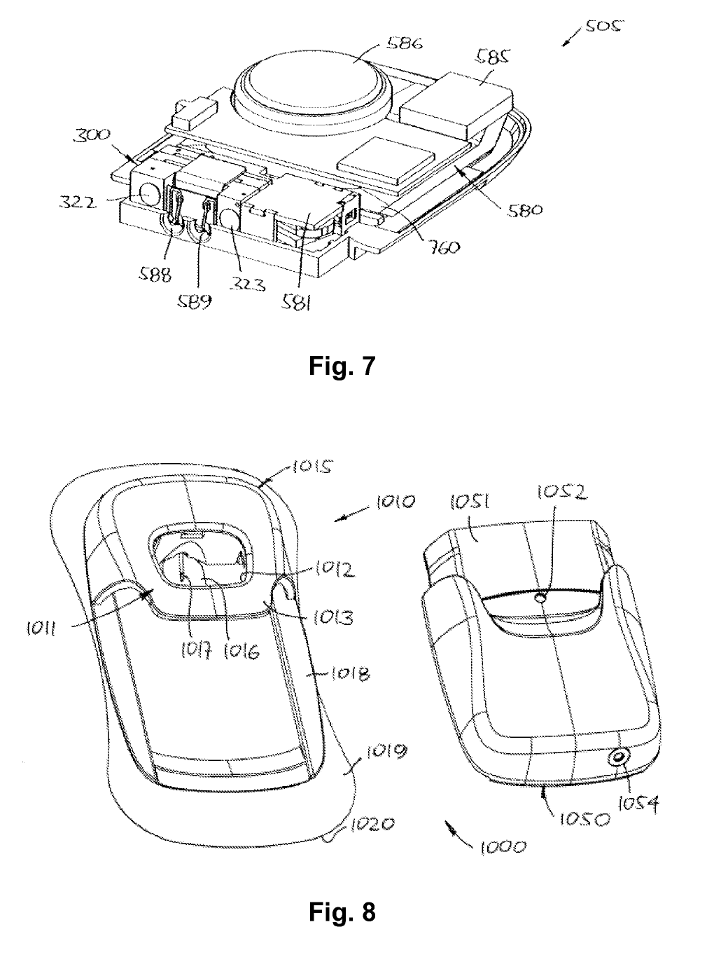

FIG. 2 shows a schematic cross-sectional view through a pump and actuator assembly of the type shown in FIG. 1, the section corresponding to a plane above the lever. Corresponding to the FIG. 1 embodiment, the assembly comprises a housing 120 for accommodating the actuator lever 130, a pair of magnets 140 as well as a pump assembly 150, the housing comprising a knife-edge member 126. The pump assembly may be of the type disclosed in FIG. 7. The actuator lever comprises first and second grooves 133, 134, a coil 136 and a contact rod 137 adapted to engage first and second contact members 128, 129 arranged on the housing. The lever further comprises a pair of conductors 138 for energizing the coil as well as a conductor 139 for the contact rod. In the shown embodiment the conductors are shown with terminal contact points, however, advantageously the three conductors are formed on a flex-print attached to the lever and connected to a structure of the device in which the actuator is mounted, the connection between the moving lever and the other structure being provided by a film hinge formed by the flex-print. The pump comprises a pump chamber 153, in which an elastic pump membrane 154 is arranged, and a bore 156 for slidingly receive and support a piston rod 151 with a convex piston head 155 engaging the pump membrane. The pump membrane is in all positions in a stretched state, the membrane thereby exerting a biasing force on the piston rod which is used to hold the actuator lever in place as described above. The pump further comprises an inlet conduit 160 with an inlet valve 161 in fluid communication with the pump chamber, and an outlet conduit 170 with an outlet valve 171 in fluid communication with the pump chamber. The valves may be of any desirable configuration, but advantageously they are passive membrane valves.

FIG. 2 shows the pump and actuator assembly in an initial state with the actuator lever in an initial position in which the contact rod 137 is positioned against the first contact member 128 which thereby serves as a stop for the lever. As indicated above, the piston rod 151 has a length which ensures that it is forced by the pump membrane into contact with the lever in its initial position. The terms “initial” and “actuated” state refers to the shown embodiment in which the actuator is used to actuate the pump to produce a pump stroke, however, although the suction stroke of the pump may be passive (i.e. performed by the elastic energy stored in the pump membrane during the pump stroke) the actuator may also be actuated in the reverse direction (i.e. from the actuated to the initial position) to actively drive the pump during the suction stroke. Thus, in more general terms the actuator is moved between first and second positions in either direction.

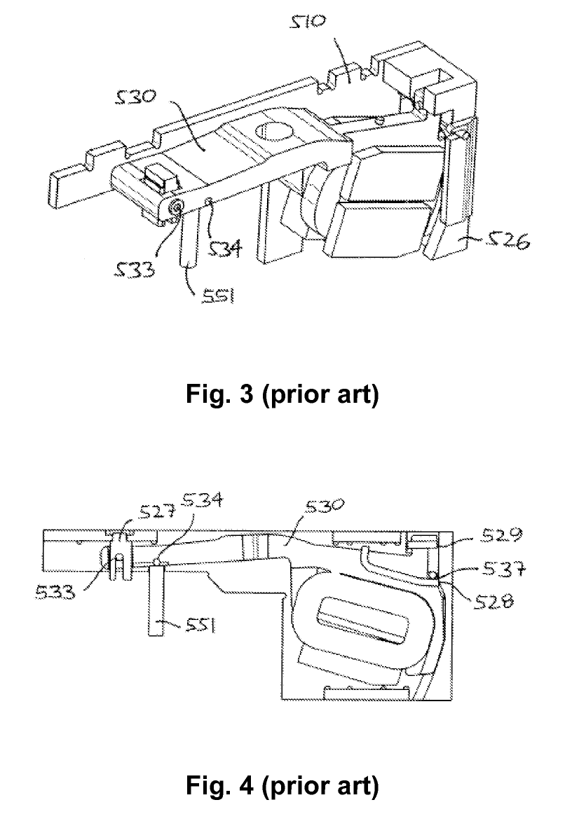

With reference to FIG. 3 a further pump actuator will be described. Although the figure is onented differently, the same terminology as for FIG. 1 will be used, the two pump actuators generally having the same configuration. In an assembled state as shown (the lower housing member not being shown for clarity reasons) a lever 530 is arranged inside a housing formed by a first housing member 510, a second housing member, and a proximal connection member 526, with the coil positioned between two pair of magnets. The lever has a proximal end comprising first and second longitudinally offset and opposed joint structures in the form of an axle rod 533 respectively a joint rod 534 arranged perpendicular to a longitudinal axis of the lever, and a distal end holding the coil member wound from a conductor. When the actuator is attached to a pump assembly (see e.g. FIG. 7) the joint rod 534 engages the substantially planar end surface of the piston rod 551, thereby forming a distal floating knife-edge pivot joint. Although the joint rod is not a “knife”, the circular cross-sectional configuration of the rod provides a line of contact between the rod and the end surface, and thus a “knife-edge” joint. Using a more generic term, such a joint may also be termed a “line” joint. Due to this arrangement a gearing of the force provided from the coil-magnet actuator to the actuation rod is realized, the gearing being determined by the distance between the two pivot joints and the distance between the proximal pivot joint and the “effective” position of the coil on the lever. As the piston rod is biased outwardly by the elastic pump membrane, the lever is held in place by the two joints and the housing in combination, the lever only being allowed to pivot relative to the first joint. The actuator further comprises a pair of rod-formed stop members 528, 529 (which may also serve as contacts) mounted on the distal end of the lever and adapted to cooperate with a rod 537 mounted in the proximal connection member.

In prior art pump assemblies and actuator systems as shown in FIGS. 1-4 a pump actuator is provided in the form of a pivoting actuator lever acting on a pump member in the form of a piston, the actuator lever providing a coil-magnet actuator with the coil being arranged on the actuator lever and the magnets being arranged on a supporting housing structure. As the actuator lever has a pivoting point at one end of the lever and the relatively heavy coil is arranged at the other end of the lever, the lever is not balanced in respect of influences from the outside, i.e. if the pump and its supporting structure is moved by external forces the lever will tend not to move with the pump but relative to the pump and thereby potentially actuate the pump, this due to the momentum of inertia of the lever.

To compensate for this the lever could be balanced with a mass counteracting the coil, however, this would only balance the lever for linear forces but not for rotational forces, in fact, such a counterweight would substantially increase the angular momentum of inertia and make the pump even more susceptible to rotational influence. Thus to perfectly balance the lever all the mass would have to be arranged corresponding to the pivoting point which indeed is not feasible.

Thus to provide an actuator system which to a high degree makes it possible to optimize the system to reduce the influence of external forces on the system, a two-member linked actuator system is provided.

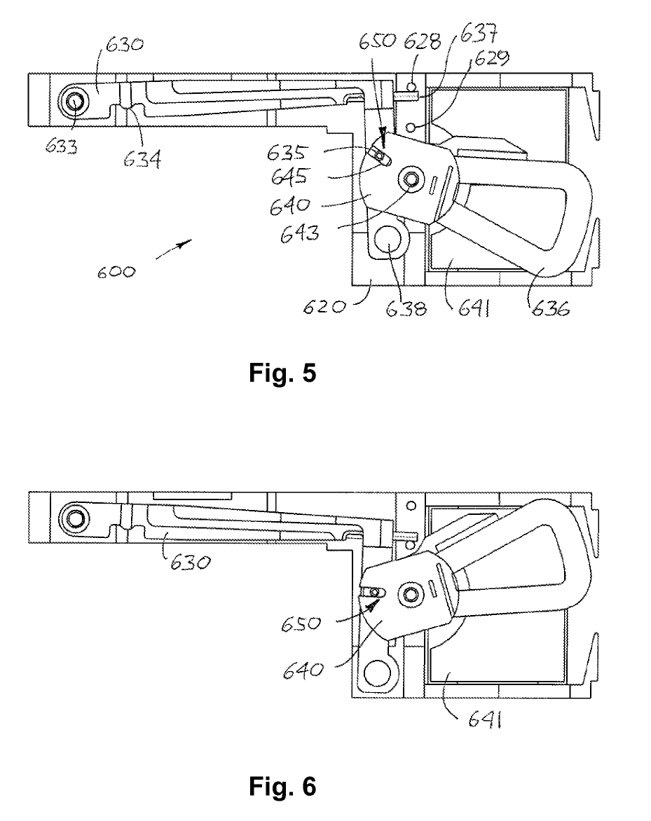

More specifically, the actuator system 600 shown in FIG. 5 comprises an actuator lever 630 and a coil-magnet actuator, the actuator comprising an actuator member 640 with a coil 636 disposed between magnets 641 (only one shown) arranged on a supporting structure 620. The system is adapted to be used with a pump comprising a pump member moveable by actuation of the actuator lever, e.g. corresponding to FIG. 1, for which purpose the actuator lever comprises a joint portion 634 (corresponding to joint pin 534) adapted to engage the member to be moved. A first pivoting joint in the form of a first axial bearing 633 is formed between the actuator lever and the supporting structure, and a second pivoting joint in the form of a second axial bearing 643 is formed between the actuator member and the supporting structure. The actuator lever and the actuator member are coupled to each other by a coupling joint 650 arranged between the first and the second pivoting joint, whereby rotation of the actuator member in a first direction causes the actuator to rotate in an opposite second direction.

This arrangement corresponds in principle to two gear wheels engaging each other. If the two gear wheels (or members) were identical they would balance each other, however, if they are not identical, but as long as they are in engagement with each other the “smaller” member having a lower momentum of inertia will to a certain degree counterbalance the “larger” member having a higher momentum of inertia, this resulting in a lever/actuator system having a lower susceptibility to external linear or rotational influences compared to a system in which a long single actuator lever was pivoting corresponding to the first pivoting joint and having a coil arranged at the same location as the actuator, e.g. as shown FIG. 4. To increase the momentum of inertia of the actuator lever it is provided with a weight 638, e.g. a metal element attached to a polymer actuator lever.

In the shown embodiment the actuator lever is moved between a first position (see FIG. 5) and a second position (see FIG. 6), the assembly comprising first and second stop means 628, 629 adapted to restrict movement of the actuator lever in the first respectively the second position. In the shown embodiment the actuator lever comprises a contact member 637 engaging the stop members which also serve as contact members, this allowing detection of actuator movement (see above). In a situation of use, the actuator system is coupled to a pump (as in FIG. 7) comprising a pump member moveable by actuation of the actuator lever via joint point, the pump comprising a flexible member in the form of a pump membrane which is stretched by the pump member when the pump member is moved between its first and second positions.

When coupled to a pump, the pump member (e.g. piston) has a first position corresponding to the first position of the actuator lever and a first resting condition of the pump, and a second position corresponding to the second position of the actuator lever and a second actuated condition of the pump, wherein the pump member by way of the pump membrane exerts a first force on the actuator lever in the first position and exerts a second higher force on the actuator lever in the second position when the pump membrane is stretched corresponding to a pump stroke.

The coupling joint 650 of the shown actuator system does not resemble a toothed engagement between two traditional gear wheels, but is in the form of a pin 635 arranged on the actuator lever and a guide slot (or “longhole”) 645 with two opposed walls in the actuator member in which the pin is arranged to slide, this allowing the position of the pin in the guide slot to determine the actual gear ratio between the actuator lever and the actuator. Depending on the orientation and configuration of the slot it is possible to design the system to have a varying gear ratio between the actuator member and actuator lever as a function of the rotational position of the actuator member and thus the actuator lever. This effect is due to the following: When the actuator member rotates an “actual” force is transmitted to the pin in a direction defined by the normal to the portion of the wall acting on the pin, however, the torque providing “rotational” force transmitted to the actuator lever is the fraction of the force which acts in the normal direction to a line through the first pivoting point and the pin. As can be seen in FIG. 5 the rotational force in the first position is smaller than the actual force whereas in the second position as shown in FIG. 6 the rotational force corresponds essentially to the actual force.

As follows from the above, when a pump assembly comprising an actuator system as shown in FIG. 5 is submitted to acceleration and the acceleration results in a rotational movement of the coil actuator member, then the force from the actuator acting on the actuator lever is smallest in the first position, this corresponding to a “high” gear ratio. As this position corresponds to the resting position of the pump this also means that the susceptibility to the pump being actuated by angular acceleration of the assembly is reduced. Indeed, this only has relevance if the system is not perfectly balanced with respect to both linear and angular acceleration, however, to achieve such a system may not be practically feasible. In respect of a desired actuation of a membrane pump by rotation of the coil actuator, the lower actuation force acting on the pump member at the beginning of an actuation does not influence the functionality of the pump assembly as the pump resistance initially is low as the pump membrane is just beginning to be stretched. As pump resistance increases due to further stretching of the pump membrane then also the gear ratio between the actuator coil and the actuator lever changes from “high” to “low”.

As follows from the above, by varying e.g. the pivot points of the two members, the mass, centre of mass, the position and configuration of the slot, it is possible to optimize the system within a desired frame of parameters in respect of efficiency and susceptibility to influence from external forces.

FIG. 7 shows a pump unit with an upper portion of the housing removed. The pump unit 505 comprises a reservoir 760, a pump assembly having a pump 300 as well as a coil actuator 581, and controller means 580 for control thereof. The pump assembly comprises an outlet 322 for connection to a transcutaneous access device and an opening 323 allowing a fluid connector arranged in the pump assembly to be actuated and thereby connect the pump assembly with the reservoir. The reservoir 760 is in the form of prefilled, flexible and collapsible pouch comprising a needle-penetratable septum adapted to be arranged in fluid communication with the pump assembly. The shown pump assembly comprises a mechanically actuated membrane pump of the type shown in FIG. 2, however, different types of pumps may be used.

The control means comprises a PCB or flex-print to which are connected a microprocessor for controlling, among other, the pump actuation, contacts 588, 589 cooperating with corresponding contact actuators on a patch unit (see below), position detectors in the actuator, signal generating means 585 for generating an audible and/or tactile signal, a display (if provided), a memory, a transmitter and a receiver allowing the pump unit to communicate with an wireless remote control unit. An energy source 586 provides energy.

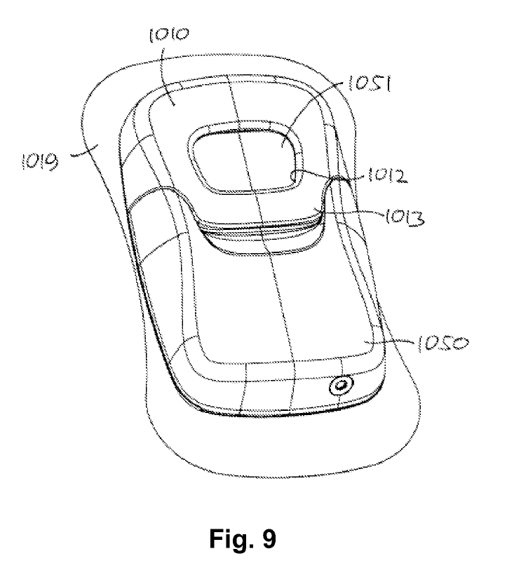

FIG. 8 shows an embodiment of a patch unit 1010 with a pump unit 1050 by its side, and FIG. 9 shows the pump unit fully but releasably attached. More specifically, FIG. 8 shows an embodiment of a medical device 1000, comprising a cannula unit 1010 of the type disclosed in applicants co-pending application WO 2006/120253, and a thereto mountable pump unit 1050. In the shown embodiment the cannula unit comprises a housing 1015 with a shaft into which a portion 1051 of the pump unit is inserted. The shaft has a lid portion 1011 with an opening 1012, the free end of the lid forming a flexible latch member 1013 with a lower protrusion (not shown) adapted to engage a corresponding depression 1052 in the pump unit, whereby a snap-action coupling is provided when the pump unit is inserted into the shaft of the cannula unit. Also a vent opening 1054 can be seen. The housing 1015 is provided with a pair of opposed legs 1018 and is mounted on top of a flexible sheet member 1019 with a lower adhesive surface 1020 serving as a mounting surface, the sheet member comprising an opening 1016 for the cannula 1017.

As appears, from the housing of the cannula unit extends a cannula at an inclined angle, the cannula being arranged in such a way that its insertion site through a skin surface can be inspected (in the figure the full cannula can be seen), e.g. just after insertion. In the shown embodiment the opening in the lid provides improved inspectability of the insertion site. When the pump unit is connected to the cannula unit it fully covers and protects the cannula and the insertion site from influences from the outside, e.g. water, dirt and mechanical forces (see FIG. 9), however, as the pump unit is detachable connected to the cannula unit, it can be released (by lifting the latch member) and withdrawn fully or partly from the cannula unit, this allowing the insertion site to be inspected at any desired point of time. By this arrangement a drug delivery device is provided which has a transcutaneous device, e.g. a soft cannula as shown, which is very well protected during normal use, however, which by fully or partly detachment of the pump unit can be inspected as desired. Indeed, a given device may be formed in such a way that the insertion site can also be inspected, at least to a certain degree, during attachment of the pump, e.g. by corresponding openings or transparent areas, however, the attached pump provides a high degree of protection during use irrespective of the insertion site being fully or partly occluded for inspection during attachment of the pump. In the shown embodiment an inclined cannula is used, however, in alternative embodiments a needle or cannula may be inserted perpendicularly relative to the mounting surface.

With reference to FIGS. 8 and 9 a modular pump system comprising a pump unit and a patch unit has been described, however, the system may also be provided as a unitary unit.

### Example

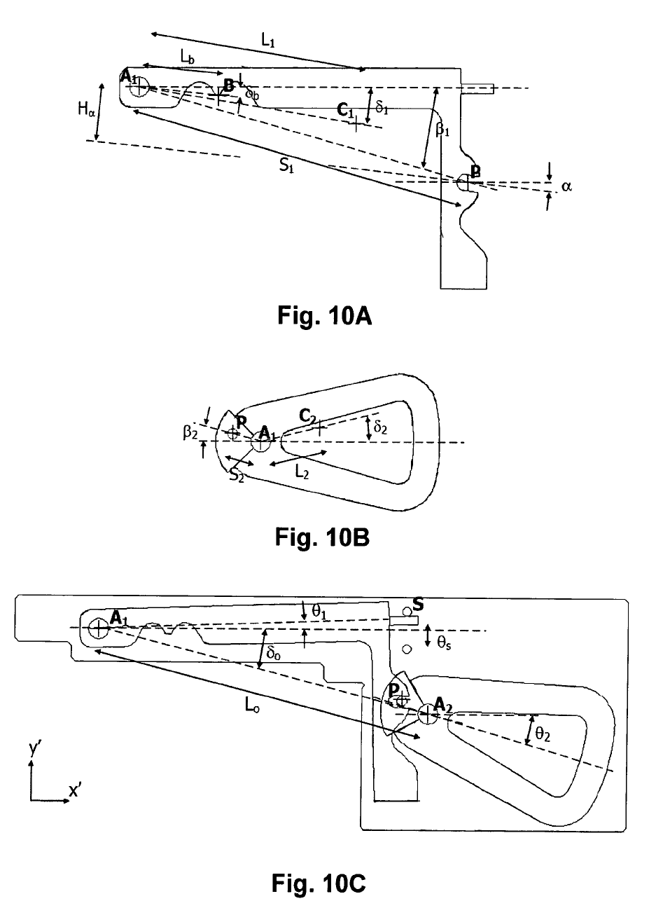

A two part arm and coil actuator system was designed and analyzed theoretically to determine the mechanical response when the system is subjected to external forces such as linear and angular accelerations, see FIGS. 10A-10C.

To simplify the problem the following approximations were made: The pump housing, arm, and coil system are stiff; the arm and coil sit tight on their axles so play can be neglected; the coil connection pin sits tight in the arm longhole so play can be neglected; friction between the mechanical parts are neglected; fictious centrifugal and coriolis forces were neglected; the dynamical equations are linearized around the rest position.

The analysis shows that it is possible in principle to design a system that is insensitive to both linear and angular accelerations in the rest position: To balance the system with respect to linear accelerations the centre of mass position for the arm and coil should be aligned according to:

M₁(C₁−A₁)=M₂G(C₂−A₂), G=−dθ₂/dθ₁

Where “₁” denotes the arm and “₂” denotes the coil, A denotes rotation point, C denotes centre of mass, M denotes mass, and G denotes gearing with θ denoting deflection angle from horizontal axis.

Further, to balance the system with respect to angular accelerations the moment of inertia of the arm and coil should be balanced according to:

I₁=G(I₂+M₂L₂Lₒ cos(θ₂+δ₂−δₒ))

Where I denotes moment of inertia around A, L₂ denotes distance |A₂C₂|, Lₒ denotes distance |A₁A₂|, δ₂ denotes angle between coil axis and A₂C₂, and δ₀ denotes angle between coil axis and A₁A₂

Indeed, the above analysis can also be used to optimize a system without striving for a system that is completely insensitive to both linear and angular accelerations in the rest position.

In the above description of the exemplary embodiments, the different structures providing the described functionality for the different components have been described to a degree to which the concepts of the present invention will be apparent to the skilled reader. The detailed construction and specification for the different structures are considered the object of a normal design procedure performed by the skilled person along the lines set out in the present specification. For example, the individual components for the disclosed embodiments may be manufactured using materials suitable for medical use and mass production, e.g. suitable polymeric materials, and assembled using cost-effective techniques such as bonding, welding, adhesives and mechanical interconnections.

1. A pump assembly (600) comprising:
an actuator lever (630),
an actuator comprising an actuator member (640) for moving the actuator lever,
a supporting structure (620),
a pump (150, 300) comprising a pump member (151) moveable by actuation of the actuator lever,
a first pivoting joint (633) formed between the actuator lever and the supporting structure, and
a second pivoting joint (643) formed between the actuator member and the supporting structure,
wherein the actuator lever and the actuator member are coupled to each other by a coupling joint (650) arranged between the first and the second pivoting joint,
whereby rotation of the actuator member in a first direction causes the actuator lever to rotate in an opposite second direction.

2. A pump assembly as in claim 1, wherein the coupling joint provides a variable gear ratio for the translation of rotational movement from the actuator member to the actuator lever.

3. A pump assembly as in claim 2, wherein the coupling joint comprises a pin (635) and a guide slot (645) in which the pin is arranged to slide, the position of the pin in the guide slot determining the actual gear ratio between the actuator lever and the actuator member.

4. A pump assembly as in claim 1, wherein the actuator lever is moved between a first position and a second position, the assembly comprising first and second stop means (628, 629) adapted to restrict movement of the actuator lever in the first respectively the second position.

5. A pump assembly as in claim 4, wherein the stop means is arranged on the supporting structure and is adapted to engage the actuator lever (630, 637) in the first respectively the second position.

6. A pump assembly as in claim 4, wherein the pump member has:
a first position corresponding to the first position of the actuator lever and a first resting condition of the pump, and
a second position corresponding to the second position of the actuator lever and a second actuated condition of the pump,
wherein the pump member exerts a first force on the actuator lever in the first position and exerts a second higher force on the actuator lever in the second position.

7. A pump assembly as in claim 6, wherein the pump comprises a flexible member (154) which is stretched by the pump member when the pump member is moved between its first and second positions.

8. A pump assembly as in claim 6, wherein the coupling joint has a first gear ratio when the actuator lever is in the first position and second lower gear ratio when the actuator lever is in the second position.

9. A pump assembly as in claim 1, wherein the actuator is a coil-magnet actuator, the coil (636) and magnet(s) (641) being arranged on the actuator member respectively the supporting structure.

10. A pump assembly as in claim 1, wherein the pump is adapted to pump a liquid between an inlet and an outlet (322) thereof, the pump member performing a pump stroke when actuated by the actuator lever.

11. A pump assembly as in claim 10, wherein the pump comprises inlet and outlet valves (161, 171) associated with the pump inlet respectively the pump outlet, and a pump chamber (153) actuated by the pump member to perform a pump stroke respectively a suction stroke.

12. A pump assembly as in claim 10, further comprising:
a reservoir (760) adapted to contain a fluid drug and comprising an outlet in fluid communication with or being adapted to be arranged in fluid communication with the pump inlet, and
a transcutaneous access device (1017) comprising a distal end adapted to be inserted through the skin of a subject, the transcutaneous access device comprising an inlet in fluid communication with or being adapted to be arranged in fluid communication with the pump outlet.

13. A pump assembly as in claim 1, further comprising a power source and processor means for controlling the actuator.
